(19) United States
(12) Patent Application Publication
(10) Pub. No.: US 2007/0233252 A1
(43) Pub. Date: Oct. 4, 2007

(54) DEVICES, SYSTEMS AND METHODS FOR TREATING INTERVERTEBRAL DISCS

(76) Inventor: Daniel H. Kim, Los Altos, CA (US)

Correspondence Address: PATTERSON & SHERIDAN, L.L.P., 3040 POST OAK BOULEVARD, SUITE 1500, HOUSTON, TX, 77056, US

(21) Appl. No.: 11/678,134

(22) Filed: Feb. 23, 2007

Related U.S. Application Data
(60) Provisional application No. 60/776,860, filed on Feb. 23, 2006.
(60) Provisional application No. 60/787,784, filed on Mar. 31, 2006.

(51) Int. Cl. A61F 2/44

(52) U.S. Cl. 623/17.11; 623/17.16

(57) ABSTRACT

Devices, systems, and methods are provided for treating intervertebral discs. In one embodiment, the systems include instruments for implanting the disc repair devices in a minimally invasive manner. The methods are directed to the minimally invasive implantation of one or more of the disc repair devices to within the intervertebral disc.

### CROSS-REFERENCE TO RELATED APPLICATIONS

This application claims benefit of U.S. Provisional Patent Application No. 60/776860, filed on Feb. 23, 2006 and U.S. Provisional Patent Application No. 60/787784, filed on Mar. 31, 2006, which applications are herein incorporated by reference in their entirety.

### FIELD OF THE INVENTION

The present invention is directed towards the minimally invasive repair of intervertebral discs.

### BACKGROUND OF THE INVENTION

The spinal column is formed from a number of bony vertebral bodies separated by intervertebral discs which primarily serve as a mechanical cushion between the vertebral bones, permitting controlled motions (flexion, extension, lateral bending and axial rotation) within vertebral segments. The normal, natural intervertebral disc is comprised of three components: the nucleus pulposus (“nucleus”), the annulus fibrosis (“annulus”), and two opposing vertebral end plates.

The two vertebral end plates are each composed of thin cartilage overlying a thin layer of hard, cortical bone which attaches to the spongy, richly vascular, cancellous bone of the vertebral body.

The nucleus is constituted of a gel-like substance having a high (about 80-85%) water content, with the remainder made up mostly of proteoglycan, type II collagen fibers and elastin fibers. The proteoglycan functions to trap and hold the water, which is what gives the nucleus its strength and resiliency.

The annulus is an outer fibrous ring of collagen fibers that surrounds the nucleus and binds together adjacent vertebrae. The fibers of the annulus consist of 15 to 25 overlapping collagen sheets, called lamellae, which are held together by proteoglycans. The collagen fibers that form each lamellae run parallel at about a 65° angle to the sagittal plane; however, the fibers of adjacent lamellae run in opposite directions from each other. As such, half of the angulated fibers will tighten when the vertebrae rotate in either direction. This configuration greatly increases the shear strength of the annulus helping it to resist torsional motion. The annulus has a height of about 10 to 15 mm and a thickness of about 15 to 20 millimeters, occupying about ⅔ of the intervertebral space.

With aging and continued stressing, the nucleus becomes dehydrated and/or one or more rents or fissures may form in the annulus of the disc. Such fissures may progress to larger tears which allow the gelatinous substance of the nucleus to migrate into the outer aspects of the annulus which may cause a localized bulge, also referred to as protrusion or herniation. In the event of annulus rupture, the gelatinous substance may escape, causing chemical irritation and inflammation of the nerve roots.

Posterior protrusions of intervertebral discs are particularly problematic since the nerve roots are posteriorly positioned relative to the intervertebral discs. Impingement or irritation of the nerve roots not only results in pain in the region of the back adjacent the disc, but may also cause radicular pain such as sciatica. Nerve compression and inflammation may also lead to numbness, weakness, and in late stages, paralysis and muscle atrophy, and/or bladder and bowel incontinence.

Progressive degeneration of the disc also leads to a reduction in disc height thereby increasing the load on the facet joints. This can result in deterioration of facet cartilage and ultimately osteoarthritis and pain in the facet joints.

The most common treatment for a disc protrusion or herniation is discectomy. This procedure involves removal of the protruding portion of the nucleus and, most often, the annular defect does not get repaired. Discectomy procedures have an inherent risk since the portion of the disc to be removed is immediately adjacent the nerve root and any damage to the nerve root is clearly undesirable. Further, the long-term success of discectomy procedures is not always certain due to the loss of nucleus pulposus which can lead to a loss in disc height. Loss of disc height increases loading on the facet joints which can result in deterioration of the joint and lead to osteoarthritis and ultimately to foraminal stenosis, pinching the nerve root. Loss of disc height also increases the load on the annulus as well. As the annulus fibrosis has been shown to have limited healing capacity subsequent to discectomy. A compromised annulus may lead to accelerated disc degeneration which may require spinal interbody fusion or total disc replacement.

Various annular defect repair techniques have been developed to occlude an aperture, whether surgically or naturally formed, within the annulus. Many of these techniques include the implantation of devices, such as patches, membranes, stents and the like, to form a barrier across the annulus aperture in order to seal or occlude the aperture and/or to prevent explant of native or prosthetic nuclear material. While an improvement over conventional suturing, these annulus implants and repair techniques are limited in their ability to provide the extent of circumferential and radial competency to the annulus for long-term success.

Accordingly, it would be highly advantageous to be able to repair a degenerating or ruptured disc in a manner which obviates the inherent risks of discectomy procedures, and which repairs and augments the annulus in a way that reduces the risk of re-herniation of the disc subsequent to repair. Additionally, it would be highly beneficial to provide a technique which allows disc repair in a minimally invasive requiring minimal steps and instrumentation to perform both annuloplasty and/or nucleus replacement procedures concurrently in a synergistic manner.

### SUMMARY OF THE INVENTION

Embodiments of the present invention provide implantable devices for repairing the intervertebral disc. The implantable disc repair devices may be configured to repair a defect in a disc annulus by retaining material (either natural or prosthetic) in the nucleus while stabilizing the defective portion of the annulus. The disc repair devices may further be configured to allow in growth of the natural tissue material therethrough. The disc repair devices may be sized to span all or a substantial portion of an annular defect. In certain variations, the devices are sized to span over an area greater than that of the defect and/or extend into one or more of the vertebral endplates, and in still other variations, extend into one or more of the vertebral bodies. As such, some of the disc repair devices are configured in a manner to bear at least part of the natural axial loads exerted on the annulus so that further deterioration of the annulus is prevented or substantially delayed. One or more of these devices may be provided along with instrumentation for implanting them in the form of a system or kit.

Embodiments of the invention further include methods directed to the minimally invasive implantation of one or more disc repair devices of the present invention at least partially within a defective area of an intervertebral disc annulus. In many applications, the subject methods involve implanting one or more subject devices between adjacent lamellae or plies of the annulus. Still yet, in certain applications, the methods involve positioning a portion of the implantable device into one or both vertebral body endplates or into the vertebral bodies themselves.

Embodiments of the present invention provide an implant delivery system for implanting a one or more disc repair devices at least partially within a defective area of an intervertebral annulus. In one embodiment, the implant delivery system is adapted to deliver the disc repair device without substantially reducing the size of the disc repair device. In another embodiment, the implant deliver system may include a dilator for dilating an opening in the annulus and a holder for holding the implantable device. In yet another embodiment, the implant delivery system may further include a cutting device for forming a space to retain the disc repair device.

In one embodiment, an implantable device for repairing a defective area of an annulus of an intervertebral disc includes a planar structure having a dimension greater than the defective area wherein at least a portion of the implantable device extends beyond the defective area upon implantation within the defective area. In another embodiment, the implantable device further includes a plug extending from the planar structure. In yet another embodiment, the device has at least one dimension that is greater than a natural disc height.

In another embodiment, a system of treating an intervertebral disc annulus includes an implantable device; a dilator for dilating an opening in the annulus; and a holder for holding the implantable device. In yet another embodiment, the system further includes a cutting device for cutting the annulus.

In another embodiment, a system of treating an intervertebral disc annulus includes an implantable device and a delivery instrument for holding the implantable device and delivering the implantable device to an opening in the annulus, wherein the delivery instrument is adapted to deliver the implantable device in its natural shape. In yet another embodiment, thee delivery instrument includes a shaft and a device holding mechanism. In yet another embodiment, the device holding mechanism includes at least two arms adapted to engage an outer perimeter of the implantable device.

In another embodiment, a method of treating a defective area of an intervertebral disc annulus situated between upper and lower vertebra comprises providing a device comprising a planar structure having a dimension greater than the defective area; positioning the device in the defective area; and lodging the device within the defective area, wherein at least a portion of the device extends beyond the defective area. In yet another embodiment, the method further includes dilating the defective area. In yet another embodiment, the method further includes providing the device with a foam material.

### BRIEF DESCRIPTION OF THE DRAWINGS

Embodiments of the invention are best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1A shows a sagittal cross-section of a spinal motion segment having a herniated intervertebral disc; FIG. 1B shows a top axial view of a portion of the inferior vertebrae and the intervertebral disc of the spinal motion segment of FIG. 1A; FIG. 1C shows the view of FIG. 1B where the herniated portion of the intervertebral disc has been removed;

FIG. 2A illustrates one embodiment of implantable intervertebral disc repair device of the present invention mounted to a tissue cutting substrate; FIG. 2B illustrates an exploded view of the device of FIG. 2A;

FIG. 3A illustrates an exemplary delivery device loaded at a distal end with the implantable device of FIGS. 2A and 2B; FIG. 3B shows an enlarged view of the distal end of the delivery device of FIG. 3A;

FIGS. 4A-4D show various acts of an exemplary method for implanting the device of FIGS. 2A and 2B using the delivery device of FIGS. 3A and 3B;

FIGS. 5A and 5B show acts of an optional annulus “pre-cut” procedure which may be performed prior to the implantation method of FIGS. 4A-4D;

FIGS. 6A and 6B show planar and side views of another embodiment of an implantable disc repair device of the present invention;

FIG. 7 illustrates a variation of the device of FIGS. 6A and 6B;

FIG. 8 illustrates an enlarged view of the distal end of another embodiment of a tissue cutting instrument useful for implanting a disc repair device;

FIGS. 9A-9C illustrate another embodiment of an optional vertebral body/end plate “pre-cut” procedure which may be performed prior to implantation of a disc repair device;

FIG. 10A illustrates another embodiment of a delivery device loaded at a distal end with the implantable device of FIGS. 6A and 6B; FIG. 10B shows an enlarged view of the distal end of the delivery device of FIG. 10A;

FIGS. 11A-11D show various acts of an exemplary method for implanting the device of FIGS. 6A and 6B using the delivery device of FIGS. 10A and 10B.

FIG. 12A illustrates another embodiment of a delivery device loaded at a distal end with the implantable device of FIGS. 6A and 6B; FIG. 12B shows an enlarged view of the distal end of the delivery device of FIG. 12A;

FIGS. 13A-13C show various acts of an exemplary method for implanting the device of FIGS. 6A and 6B using the delivery device of FIGS. 12A and 12B;

FIG. 14A illustrates an embodiment of a polymer-coated ring-type implant; FIG. 14B illustrates the ring-type implant of FIG. 14A configured with a foam plug; and FIG. 14C illustrates an embodiment of a polymer-coated plate-type implant configured with a foam plug;

FIGS. 15A-15C illustrate various components of an exemplary implant delivery system of the present invention, where FIG. 15A shows a dilator, FIG. 15B shows an implant holder, and FIG. 15C shows a pre-cutter;

FIGS. 16A-D illustrate a manner of using the dilator of FIG. 15A;

FIGS. 17A-17F illustrate various acts for implanting the device of FIG. 14A using the system of FIGS. 15A-15C, where FIGS. 17A-B show the act of dilating the annulus, FIGS. 17C-F show the act of precutting the annulus, FIGS. 17G-J show the act of positioning the implant at a target site within the annulus, FIGS. 17K-O show the act of rotating the implant at the target site, FIGS. 17P-S show the act of pushing the implant off the implant holder, and FIGS. 17T-U show the implant fully implanted at the at the target site;

FIGS. 18A-E illustrate various acts for implanting the device of FIG. 14B using the system of FIGS. 15A-15C, where FIGS. 18A-B show the act of rotating the implant at the target site, FIGS. 18C-D show the act of pushing the implant off the implant holder, and FIG. 18E shows the implant fully implanted at the at the target site; and

FIGS. 19A-19H illustrate various acts for implanting the device of FIG. 14C using the system of FIGS. 15A-15C, where FIGS. 19A-B shows the act of rotating the implant at the target site, FIGS. 19C-D illustrate the act of pushing the implant off the implant holder, FIGS. 19E-G show the implant initially implanted within the target site with the pre-attached sutures in a taut condition and the subsequent expansion of the implant upon cutting the sutures; and FIG. 19H shows the implant fully implanted at the at the target site.

### DETAILED DESCRIPTION OF THE INVENTION

Before the implantable disc repair devices, systems and methods are described, it is to be understood that the present invention is not limited to particular embodiments described and shown in the Figures, and as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. For example, in this description and the following claims, the terms “anterior”, “posterior”, “superior” and “inferior” are defined by their standard usage in anatomy, i.e., anterior is a direction toward the front (ventral) side of the body or spinal motion segment; posterior is a direction toward the back (dorsal) side of the body or functional spine unit; superior is upward toward the head; and inferior is lower or toward the feet.

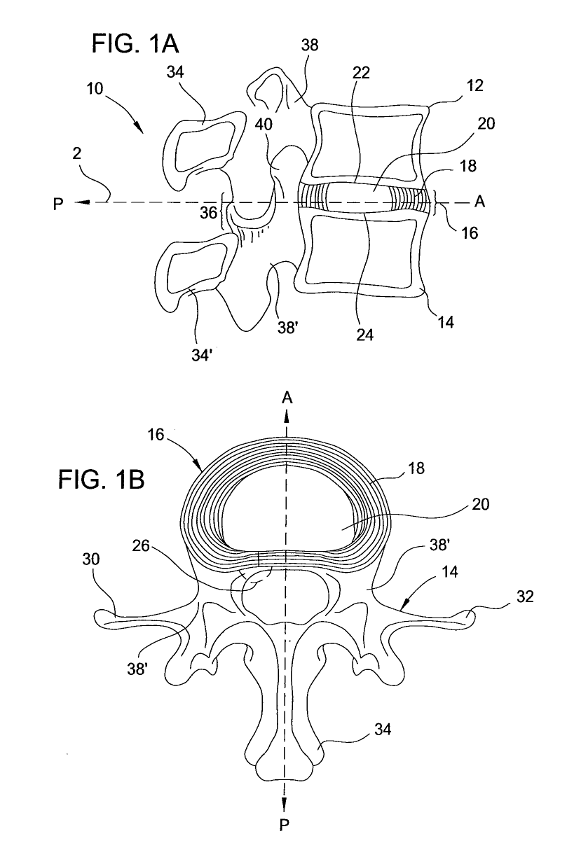

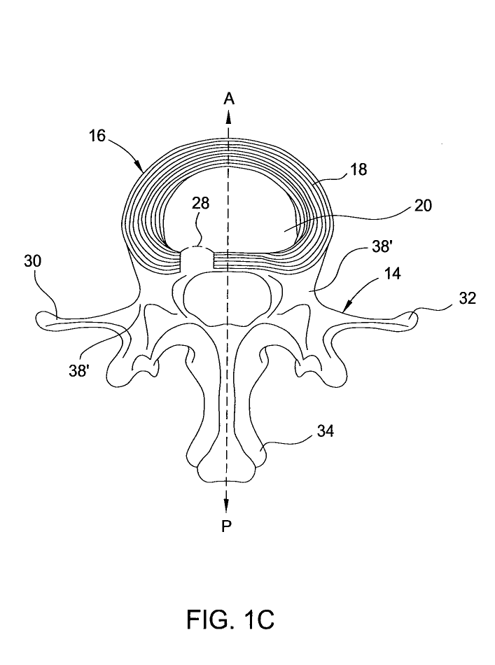

Referring now to FIGS. 1A and 1B, the general anatomy of a spinal motion segment 10 is illustrated. Axis 2 shows the anterior (A) and posterior (P) orientation of the spinal motion segment within the anatomy. A spinal motion segment includes the bony structures of two adjacent vertebrae (superior vertebral body 12 and inferior vertebral body 14), the intervertebral disc 16 (including the annulus fibrosis 18, the nucleus pulposus 20, and endplates 22, 24 of the vertebrae), and the ligaments, musculature and connective tissue (not shown) connected to the vertebrae. Intervertebral disc 16 substantially fills the space between the two vertebral bodies to support and cushion them, and permits movement of the two vertebral bodies with respect to each other and other adjacent spinal motion segments. Extending posteriorly from each of vertebral bodies 12 and 14 are left and right transverse spinous processes 30, 32 and a posterior spinous process 34, 34′. The vertebral bodies also include facet joints 36 and pedicles 38, 38′ that form the neural foramen 40.

As discussed above, progressive degeneration of the disc results in disc height loss where the superior vertebral body 12 moves inferiorly relative to the inferior vertebral body 14. Ultimately, this may result in herniation of the disc, as illustrated by herniated segment 26, shown in phantom in FIG. 1B, which protrudes beyond the posterior border of annulus 18. FIG. 1C illustrates the disc defect or void 28 created by a discectomy procedure in which the herniated portion 26 of annulus 18 and nucleus 20 has been removed. Such a discectomy procedure may be performed, but is not required to be performed, prior to use of the devices and practice of the methods of the present invention.

Embodiments of the present invention are directed to repairing the intervertebral disc and for treating or preventing degeneration and/or further herniation of the intervertebral disc. This may be accomplished by implantation of one or more of the subject devices within at least a portion of the confines of the disc annulus space, and typically within at least a portion of a defective portion of the annulus. The subject devices may be sized such that they have a planar dimension which extends beyond the defective portion of the disc annulus and/or beyond the confines of the annular space when implanted. The generally planar configuration of the disc repair devices allows them to be positioned within an intra-annular space (i.e., between two adjacent lamellae or an inter-lamellar space), or within a sub-annular space (i.e., between the innermost lamella and the outer aspect of the nucleus), or within a natural or interventional void in the annulus. For example, upon implantation, a disc repair device may extend within healthy annular tissue and/or to within one or both of the vertebral body endplates or within one or both of the intervertebral bodies in between which the disc is situated.

The disc repair devices may have a fixed size and shape which does not vary prior to, during or after implantation of the devices within the spinal motion segment. The fixed size/shape aspect of the device allows the use of more rigid materials which may provide greater durability and reliability in the long run. Although this disclosure primarily illustrates and describes such fixed shape/size devices, in certain embodiments, the devices may have a flexible or bendable, or otherwise expandable or compressible, construct such that the size and/or shape of the device is changeable between a lower-profile state to a higher profile state, and/or visa-versa, to enable minimally invasive delivery to the intra-annular or sub-annular implant site. Various exemplary embodiments of such bendable or flexible implantable devices are disclosed in U.S. patent application Ser. No. 11/271,525, filed on Nov. 10, 2005, incorporated herein by reference.

The disc repair devices preferably have dimensions sufficient to bridge across void 28 (FIG. 1C) upon implantation (or in a fully deployed or expanded state with respect to relevant embodiments). Typically, the axial (i.e., along the axis of the spine) or height dimension of a subject disc repair device is at least that of the natural height of a healthy disc and is thus in the range from about 3 mm to about 16 mm; however, in certain embodiments the height of the device may be less than the height of the disc. Typically, the dimension of the subject disc repair devices transverse to and in a lateral direction of the spine's axis expands across the entirety of the defect is in the range from about 2 mm to about 14 mm, and most typically from about 4 mm to about 10 mm. For purposes of this discussion, the dimension of a subject disc repair device, in a fully implanted position, which runs along or parallel to the spine's axis is referred to as the “axial dimension”, and the dimension which runs transverse to the axial dimension is referred to as the “transverse dimension”.

In one embodiment, the subject disc repair devices may have configurations which retain material within the nucleus while allowing for tissue in growth. The configurations may also allow for the delivery and implantation of a prosthetic material to within either or both the annulus and nucleus subsequent to implantation of the disc repair device(s). It must be noted that the devices and materials may be implanted in any order or simultaneously. For example, the disc implant devices may provide scaffolding for promoting tissue in growth and/or allowing passage of the prosthetic implant material to within the nucleus as well as to within voids within the annulus not yet occupied by the disc repair device. The scaffolding may take the form of a frame having a planar configuration having apertures or which may be partially or wholly porous, or may be configured as a mesh, webbing, fabric or an arrangement of struts having one or more openings therein to allow for the passage of in growth.

Various exemplary embodiments of the disc repair devices and disc repair methods of the present invention are now described in greater detail; however, such description is not intended to be limiting but exemplary of the present invention. Any combination of features, materials, functions and physical characteristics described above may be applied to each of the devices and/or materials of the present invention.

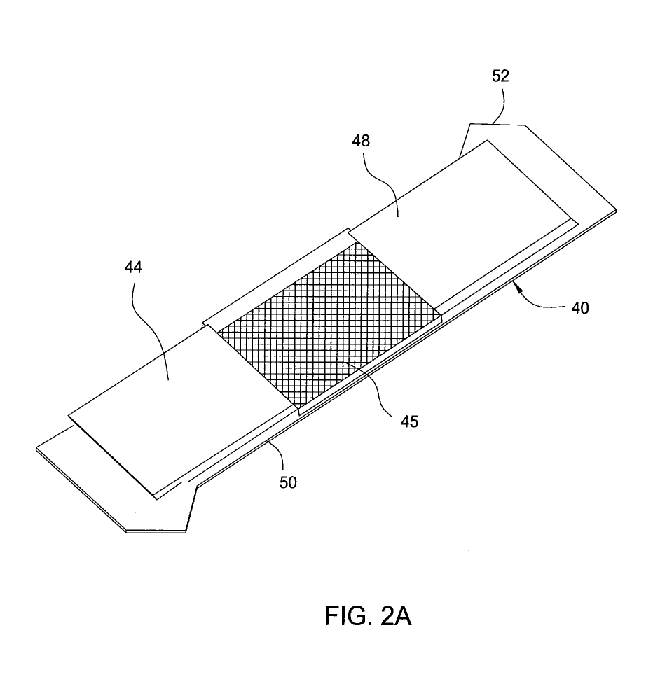

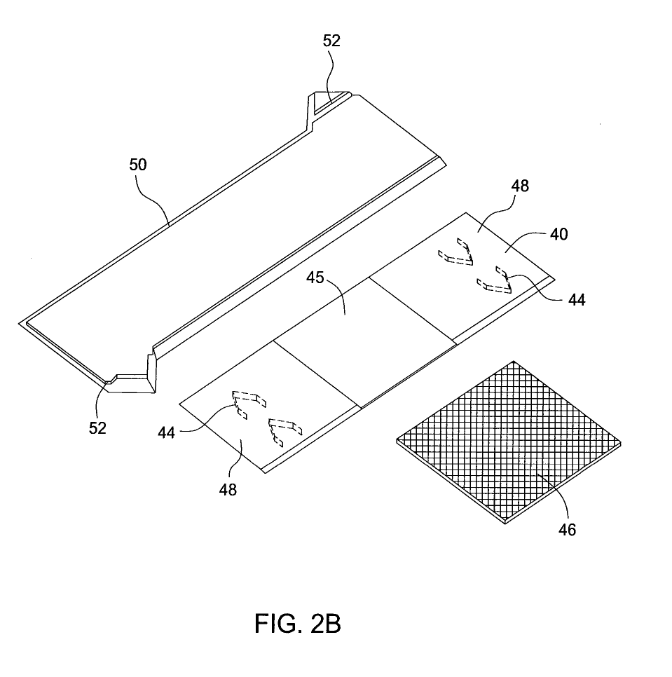

FIGS. 2A and 2B illustrate one embodiment of a disc repair device 40 of the present invention. In FIG. 2A, the intervertebral disc repair device 40 is shown mounted to a tissue cutting substrate 50. FIG. 2B illustrates an exploded view of the disc repair device 40. Device 40 has a thin planar structure which is designed to be implantable within an intra-annular space, e.g., inter-lamellarly (between two adjacent lamellae), to bridge a disc defect or void 28. While device 40 is shown having a rectangular shape, any suitable shape (e.g., square, elliptical, oblong, circular, etc.) which accomplishes the objectives of the invention may be employed. Depending on the length of the disc repair device 40, it may be straight (if shorter) or have a radius of curvature along its length (if longer) which matches that of the intra-annular circumference.

Each disc repair device 40 may have a central portion 45 flanked by end portions 48. The central portion 45 may be sized such that, when implanted, is positioned within disc defect 28. Central portion 45 may have a mesh configuration or include a plurality of openings or apertures which extend through the thickness of disc repair device 40 to allow for in growth therethrough and/or for the passage of an implant material as mentioned above. A polymer coating or overlay 46 may be provided on or over central portion 45 and may function as a therapeutic agent carrier, inhibit expulsion of nuclear material, and/or promote in growth. Further, the implantable devices 40 or portions thereof may be impregnated, coated or otherwise delivered with one or more therapeutic agents, including but not limited to, drugs (e.g., analgesics, antibiotics, steroids, etc.), growth factors, extracellular matrices (ECMs), etc. which may be dispersed in a regulated or time-released fashion.

Each end portion 48 may have one or more extendable anchors 44. Anchors 44 may be formed by cut outs within the disc repair device 40 and remain connected so as to be hinged and flarable or biased from the disc repair device 40 to function as barbs once operatively positioned within the annulus. As with the entirety of disc repair device 40, anchors 44 may be fabricated from a super elastic memory material which is activated by body temperature to achieve a flared condition subsequent to implantation. Alternatively, anchors 44 may be naturally biased in outward or operative position and held flush with disc repair device 40 until their extension is desired. The anchor cut-out 44 and aperture patterns of the disc repair device 40 may be formed by electro-discharge machining (EDM), laser cutting, injection molding, photo-chemical etching (PCE), a casting process or by other suitable means from a relatively thin sheet of material, e.g., having a sheet thickness from about 0.1 mm to about 4 mm.

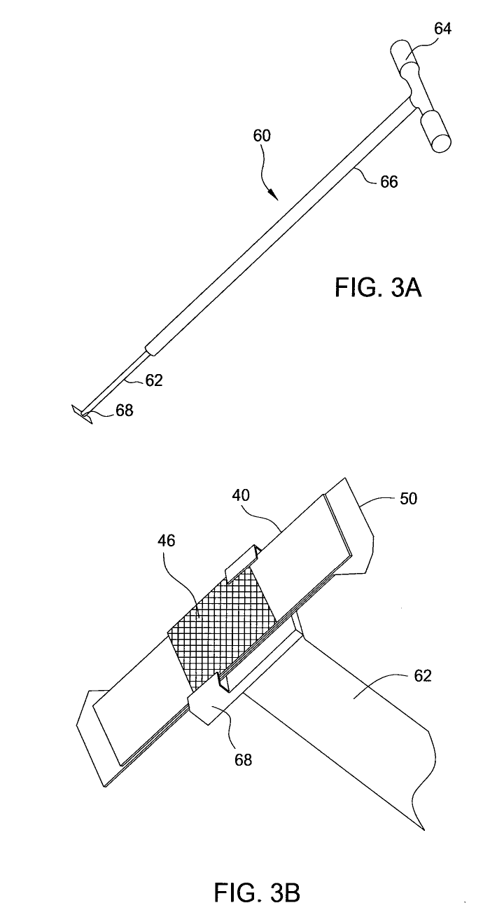

In the illustrated embodiment, the disc repair device 40 is configured to be mounted or carried by a cutting substrate 50. The cutting substrate 50 may be more or less rigid than the disc repair device 40 and has height and length dimensions which are generally equal if not a bit greater than those of the disc repair device 40. In one embodiment, the cutting substrate 50 includes bladed extensions 52 at each end thereof where the bladed extension(s) at one end extend facing in the opposite direction as the bladed extension(s) at the other end. However, the cutting substrate 50 need not have such extensions but may have edges, particularly at its distal end, which are sharp and configured to cut through tissue and/or bone. When used in conjunction with a delivery or implantation tool (such as one described below with reference to FIGS. 3A and 3B), the cutting substrate 50 is employed to carry the implantable device 40 and to create or cut openings or slits about the implant site into which the disc repair device 40 is to be positioned. In another embodiment, the disc repair device 40 may be provided with sharp cutting edges or with bladed members at its ends which function similarly to those of substrate 50 such that the cutting substrate 50 is not required.

FIGS. 3A and 3B illustrate an exemplary tool or instrument 60 suitable for implanting a disc repair device within an intervertebral disc. FIG. 3A shows the instrument 60 loaded with the disc repair device 40 shown in FIG. 2A. In one embodiment, the instrument 60 includes an elongated shaft 62 having a handle portion 64 at a proximal end and a implant holding mechanism 68 for carrying the disc repair device 40. The implant holder 68 carries the cutting substrate 50 and releasably holds the implantable disc repair device 40 flush against the cutting substrate 50. In this position, the cutting substrate 50 holds the anchors 44 flush with the cutting structure 40 in an unbiased condition. Alternatively, where planar the disc repair device 40 is itself bladed or otherwise configured to cut tissue and/or bone, the cutting substrate 50 is not needed, and the implant holding mechanism 68 is configured to releasably rotate and hold the disc repair device 40 alone. In another embodiment, the delivery instrument 60 may further include a tubular outer sheath or shaft 66 within which the elongated shaft 62 is translatable. The distal end of outer sheath 66 may be configured and dimensioned to abut the outer surface of a disc annulus 18 about the entry site into the defective portion in order to properly align the implant device 40 within the entry site and to stabilize the delivery instrument 60 during the implantation process.

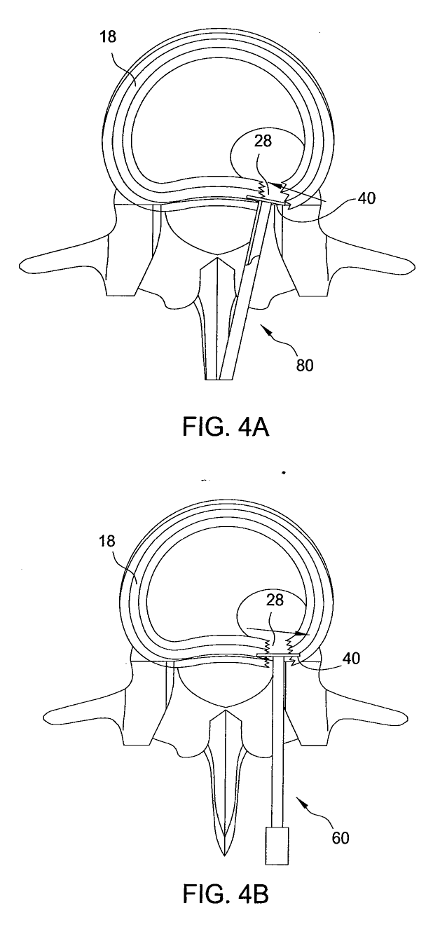

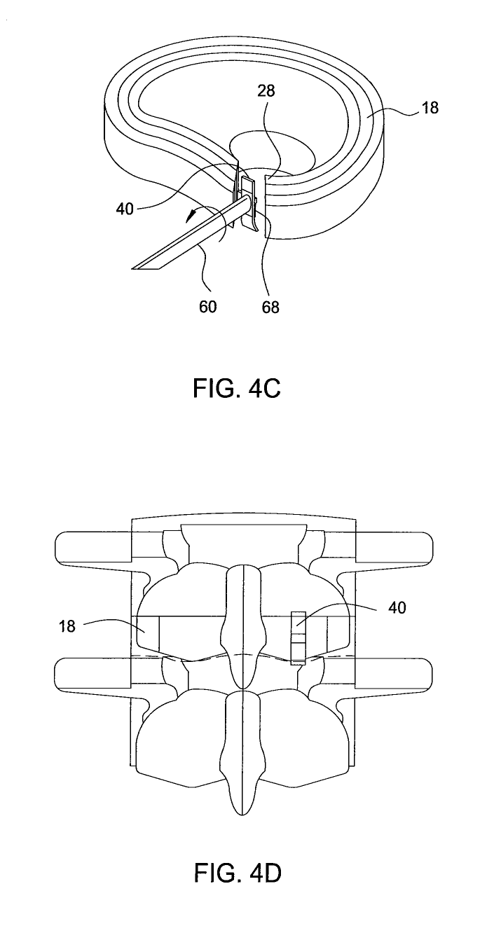

Various steps or acts of a method of implanting a disc repair device 40 using the delivery instrument 60 are illustrated in FIGS. 4A-4D. The disc repair device 40 is operatively held by or pre-loaded onto the cutting structure 50, which is affixed to the implant holding mechanism 68 of the delivery instrument 60 (as illustrated in FIGS. 3A and 3B). Upon accessing the outer annulus 18, the disc repair device 40 is inserted within annulus 18 through a void or opening 28, which may be a natural opening caused by the defect or previously formed by removal of the annular tissue. As illustrated in FIG. 4A, with the long axis of disc repair device 40 positioned substantially transverse to the axis of the spine, one end (the left end) of the device 40 is initially maneuvered through the void 28 to penetrate into the annular tissue, such as between adjacent lamellae. This step may be facilitated by using the cutting substrate 50 to gently separate or cut between the lamellae into which the device 40 is to reside. Then, the device 40 is again maneuvered to position the other side (the right side) within the annular tissue such that the disc repair device 40 straddles across defect opening 28, as illustrated in FIG. 4B. Next, the implant holding mechanism 68 is rotated about its longitudinal axis, as illustrated in FIG. 4C, in a direction whereby the bladed extensions 52 of the cutting substrate 50 are caused to dissect the annulus tissue 18 and, depending on the axial dimension of the disc repair device 40, cut into the disc's end plates and/or the vertebral bodies. In one embodiment, the disc repair device 40 may be rotated about a quarter turn (about 90°) or until its axial dimension is operatively positioned such that ends of the device 40 are securely positioned, e.g., within the vertebral end plates. In another embodiment, the disc repair device 40 may be rotated a full turn (about 360°) if necessitated or desired, but in any case, until its ends are in the caudal and cephalad positions. The implant holder 68 is then adjusted or activated to release the disc repair device 40 from the delivery instrument 60. In one embodiment, the implant holder 68 is adapted to release the disc repair device 40 by rotating the implant holder 68 in an opposite direction as the direction of rotation of the disc repair device 40 during implant. After release, the delivery instrument 60 and the cutting structure 50 are removed from the surgical site. FIG. 4D illustrates the disc repair device 40 lodged within the intervertebral disc. After removal of the cutting structure 50, anchors 44 of the disc repair device 40 are allowed to flare (either by their release from an unbiased position or due to activation by body heat) and penetrate into the surrounding tissue/bone further ensuring against the disc repair device's 40 migration from the implant site.

While one implant device 40 is typically sufficient, more than one and as many as eight or more devices may be implanted in a stacked arrangement, where at least one lamella lies between adjacently implanted devices 40. If needed or desired, the procedure described with respect to FIGS. 4A-D is repeated as necessary for the selected number of devices to be implanted, with each successive implant being inserted in an inter-lamellar layer that is more proximal (towards the outer circumference of the annulus) than the one before.

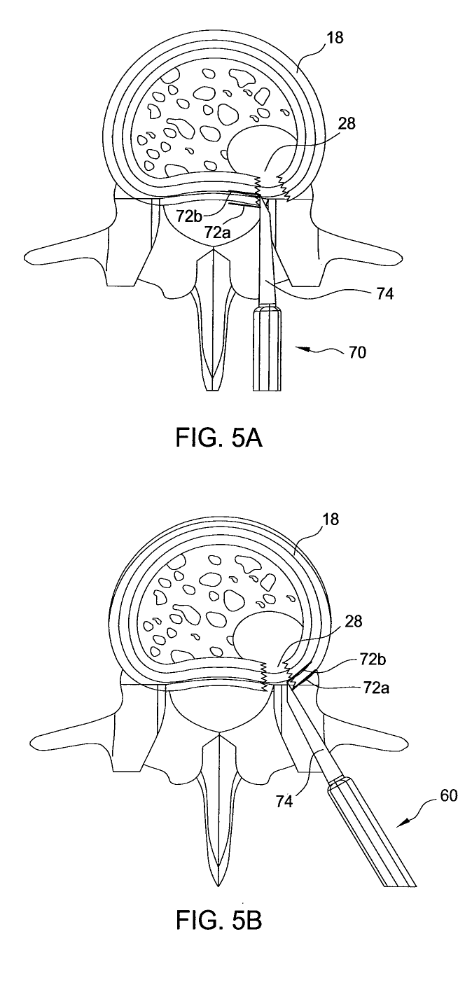

An optional set of steps may be performed prior to the implantation procedure just described in order to “pre-cut” the annulus openings or slots into which the ends of the disc repair device 40 are initially positioned prior to rotation of the device 40 into its final implanted position (i.e., where the device ends are positioned in caudal/cephalad positions). With reference to FIGS. 5A and 5B, this preliminary procedure involves the use of a tissue cutting instrument 70 having a shaft 74 and laterally extending, spaced-apart arms 72a, 72b at a distal end thereof. At least the most distally positioned arm 72b has sharp or bladed edges to cut into annulus tissue and/or separate the lamellar layers from each other. The more proximally positioned arm 72a may also have a bladed configuration to function similarly and/or may be configured for atraumatic abutment against the outer annulus surface, as illustrated. The spacing between the arms 72a, 72b may be fixed or adjustable to reach deeper lamellar layers with the distal arm 72b. Additionally, the rotational position of the arms may be adjustable whereby the distal arm 72b is rotatable relative to proximal arm 72a to provide more flexibility and control when pre-cutting tissue. Alternatively, arms 72 may simply be moved in an up-down manner to cut tissue.

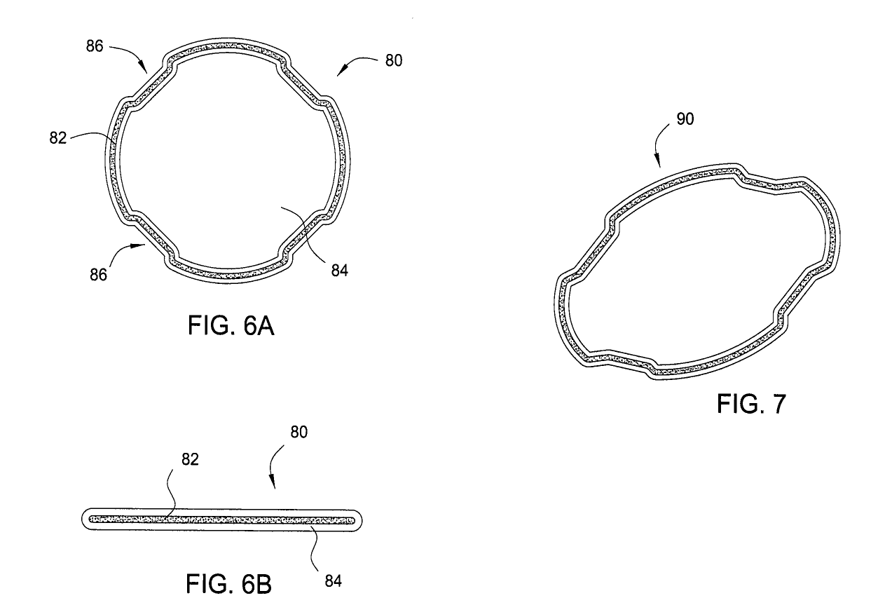

Referring now to FIGS. 6A and 6B, there are planar and side views, respectively, of another embodiment of an implantable disc repair device 80 of the present invention. The device 80 includes a frame 82 and a material 84 which is held in a relatively taut state by the frame 82 and expands across the area defined by the frame 82 to define a planar implant. The frame 82 may be made of a flexible wire such as NITINOL wire, a semi-rigid or rigid metal, or polymer wire having a diameter from about 0.05 mm to about 2 mm and more typically from about 0.1 mm to about 1 mm. The material 84 may be a polymer or other material that provides the in-growth and/or through-put characteristics as described above. The device 82 may have any suitable shape (e.g., circular, oval, elliptical (see FIG. 7), rectangular, etc.) provided that the axial and transverse dimensions (as defined above) are sufficient to repair the defective annulus. For example, the major axis of the disc repair device 90 of FIG. 7 may be employed as the axial dimension upon implant or as the transverse dimension upon implant depending on such factors as natural disc height and defect dimensions.

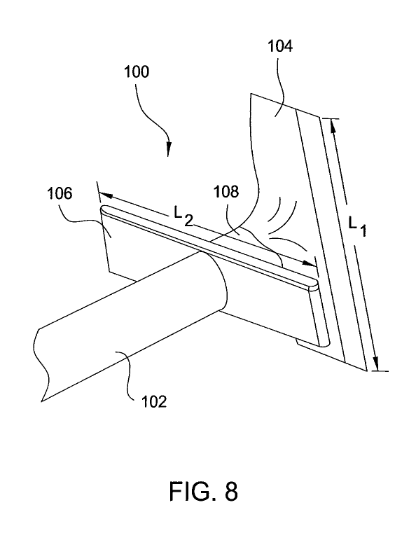

As illustrated, disc repair devices 80 and 90 have atraumatic edges; however, these configurations may also be equipped with bladed edges so as to facilitate penetration into the annulus as well as the vertebral bodies and end plates. In either case, the annulus and/or vertebral bodies/endplates may be pre-cut prior to implantation of these devices. To this end, the tissue cutting instrument 70 of FIGS. 5A and 5B may be used. Where a more robust tool is necessary, particularly for carving into the vertebral bodies and/or endplates, the tissue cutting instrument 100 of FIG. 8 may be more suitable.

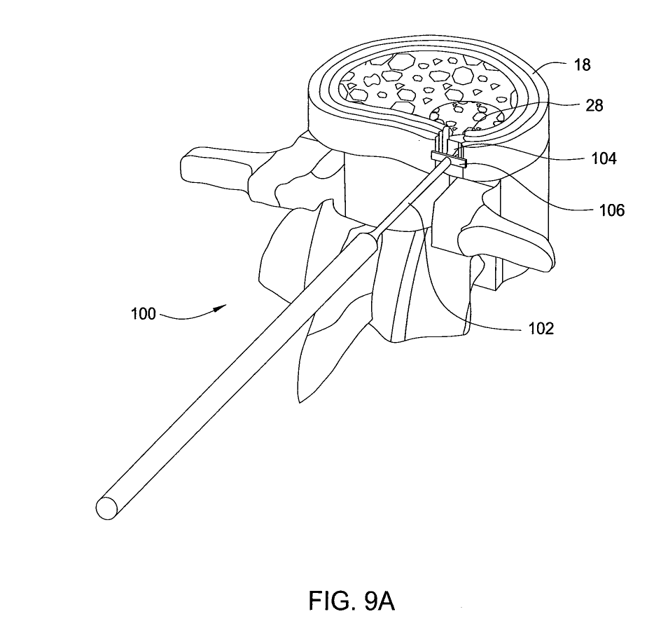

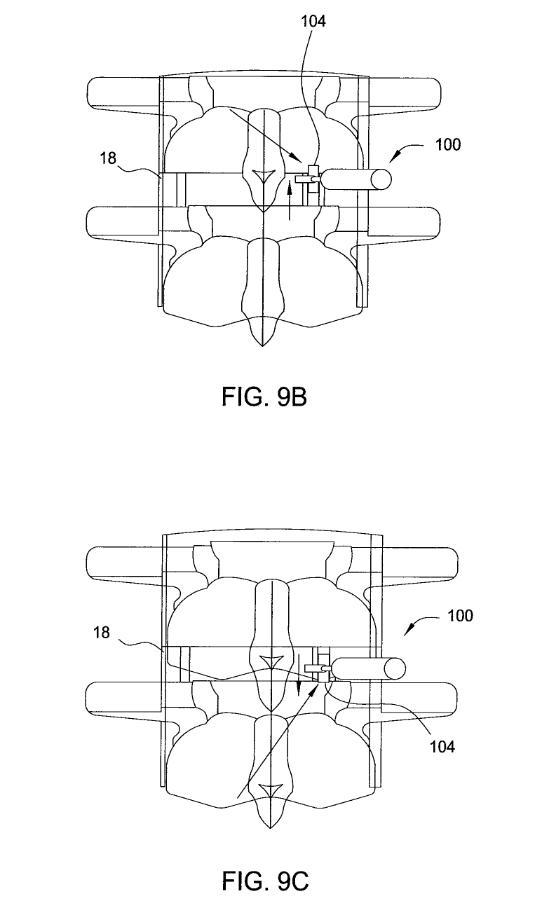

In FIG. 8, the cutting instrument 100 includes bladed member 104 positioned at a distal end of an inner shaft 108. At the distal end of outer shaft 102 and positioned proximally of bladed member 104 is a guide member 106. Both members 104, 106 have elongated configurations whereby the length (L₁) of bladed member 104 is small enough to fit through the annulus void or defect 28 when positioned parallel to the spinal axis; while the length (L₂) of guide member 106 is greater than the opening of defect 28 in order to abut against the outer surface of annulus 18 and bridge across defect 28, as illustrated in FIG. 9A. The inner shaft 108 is rotatably and translationally movable relative to the outer shaft 102 to adjust the spacing between the bladed member 104 and the guide member 106 to accommodate varying numbers of lamellar layers therebetween.

As shown in FIG. 9A, to use cutting instrument 100, upon positioning at the opening of the defect 28, the inner shaft 108 is advanced distally thereby inserting bladed member 104 into the defect 28. Upon the bladed member 104 reaching the desired depth within the annulus 18, the outer shaft 102 may be advanced to slightly compress the guide member 106 against the outer wall of annulus 18 as a means of stabilizing the instrument 100. The shaft 108 may be rotated relative to shaft 102 to cut the adhesion between the lamellar layers and/or may be moved in an up-down motion as illustrated in FIG. 9B-C to penetrate into opposing end plates. The blade member 104 may be penetrated as deeply as necessary into the vertebral bodies to achieve the desired cut-to-cut distance, which distance may be about equal to or less than that of the axial dimension of the disc repair device to be implanted. Once the space to be occupied by the repair device is sufficiently formed, the cutting instrumentation 100 is removed from the surgical site. As with any of the instruments described herein, a scope may be provided at the distal end of the cutting instrument 100, e.g., at either or both the distally facing ends of members 104 and 106, to facilitate the cutting procedure.

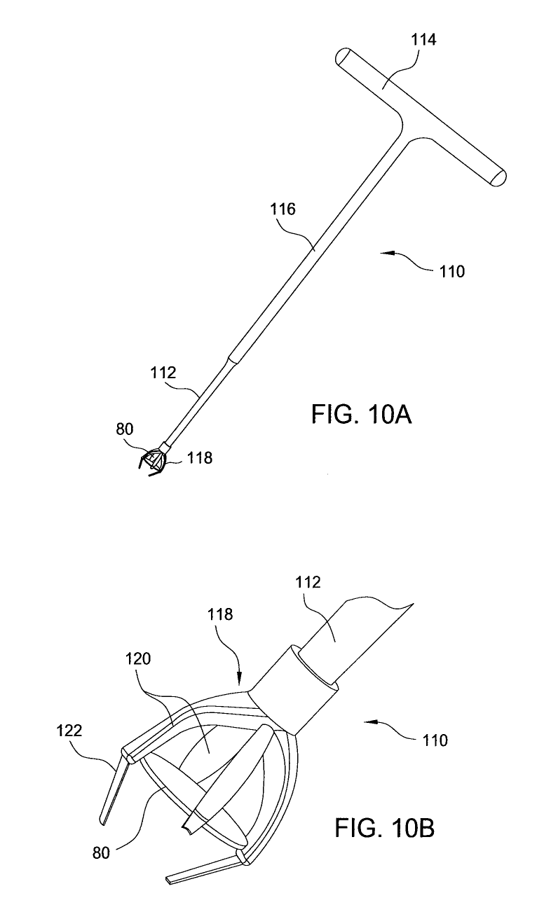

FIGS. 10A and 10B illustrate another embodiment of a delivery instrument 110 suitable for implanting disc repair devices such as those shown in FIGS. 6 and 7 within an intervertebral disc. The instrument 110 includes an elongated shaft 112 having a handle portion 114 at a proximal end and an implant holding mechanism 118 at a distal end for releasably holding the implantable disc repair device 80. The instrument 110 may further include a tubular outer sheath or shaft 116 within which elongated shaft 112 is translatable. The distal end of outer sheath 116 may be configured and dimensioned to abut the outer surface of a disc annulus about the entry site into the defective portion in order to properly align the implant device within the entry site and to stabilize the delivery instrument 110 during the implantation process.

The implant holding mechanism 118 includes two or more legs 120 configured to hold the disc repair device 80 where the planar surface is positioned transverse to the axis of the insertion path into the disc annulus. To facilitate engagement by the arms 120, the disc repair device 80, and particularly its frame 82, may be recessed or keyed along its length (see reference 86 in FIG. 6A) to engage the legs 120. To facilitate insertion into the defect 28, the legs 120 may have inwardly extending fingers 122 to provide a distally tapered configuration.

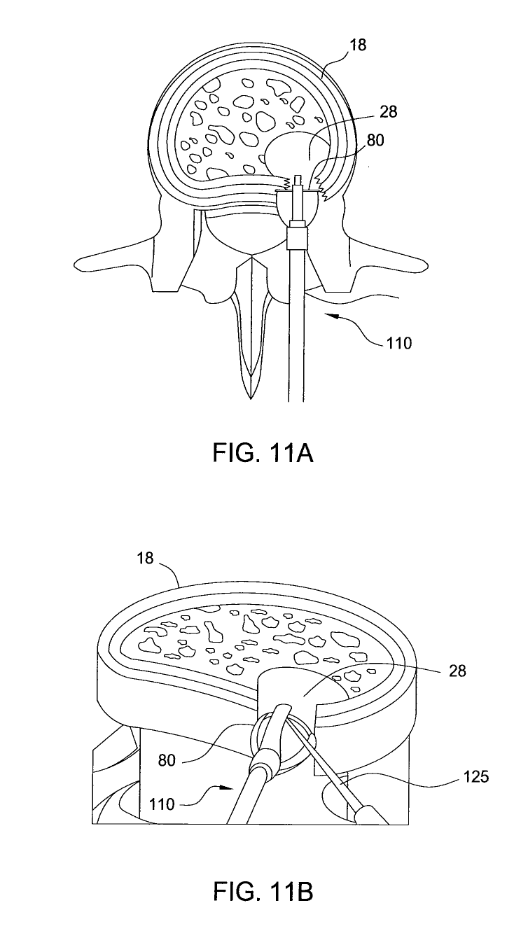

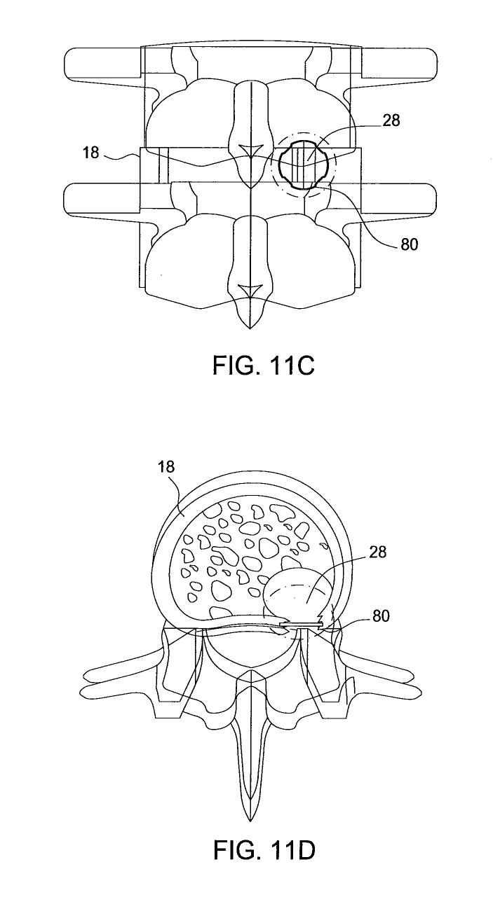

Various steps or acts of a method of implanting a disc repair device by use of the delivery instrument 110 are illustrated in FIGS. 11A-11D. After surgical access is made, the disc repair device 80, operatively held by or pre-loaded onto the implant holding mechanism 118 of the delivery instrument 110 (as illustrated in FIGS. 10A and 10B), is inserted within annulus 18 through void or opening 28, which may be a natural opening caused by the defect or formed by removal of a portion of the annular tissue. Where the disc repair device 80 has a planar dimension (e.g., diameter) greater than the size of the defect, as illustrated in FIG. 11A, the inwardly facing fingers of 122 of the device holder 118 are used to gradually expand the passage to the implant site so that the disc repair device 80 may be advanced to the implant site. Upon insertion to the desired depth within annulus 18 (or to the pre-cut space if one has been formed), the disc repair device 80 is caused to be released by the implant holding mechanism 118. Release may be accomplished by the slight radial expansion of the arms 120 or by use of another tool integrated within or separate from the delivery instrument 110 to push the disc repair device 80 off of the legs 120. As illustrated in FIG. 11B, a separate tool 125 is used to remove or release the device 80 from the grasp of the holding mechanism 118. The freed implant device 80 then readily inserts within the pre-cut space within the disc endplates, as illustrated in FIGS. 11C and 11D.

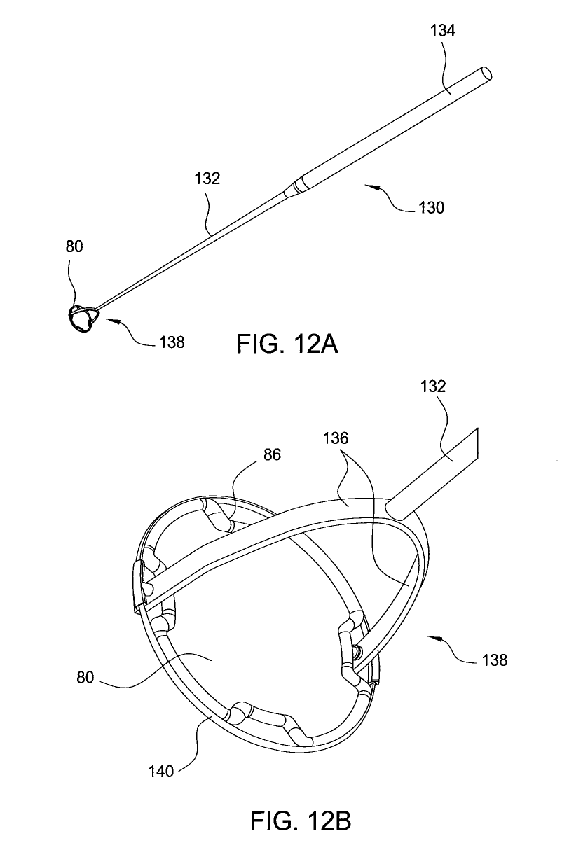

FIGS. 12A and 12B illustrate another embodiment of a delivery instrument 130 suitable for implanting disc repair devices such as those shown in FIGS. 6 and 7 within an intervertebral disc. The delivery instrument 130 includes an elongated shaft 132 having a handle portion 134 at a proximal end and an implant holding mechanism 138 at a distal end for releasably holding an implantable disc repair device 80. The holding mechanism 138 includes two diametrically opposing legs 136 pivotally attached to an implant holder 140 which is rotatable about an axis perpendicular that defined by shaft 132. In the illustrated embodiment, the implant holder 140 has a ring configuration having a rimmed internal diameter sufficient to hold the frame of the implant device 80 in frictional engagement within its perimeter. Here, the holder 140 is illustrated as a closed ring, however, the ring need not be closed, thereby allowing it to be more easily flexed—the advantages of which are illustrated in the discussion below. For example, the ring may have a slit or extend less than 360°. Alternatively, the holder 140 may comprise two opposing segments which grasp the implant device on opposing sides. Further, the holder 140 may have a shape which matches that of the implant device to be delivered. For example, a holder having a complete, open or segmented elliptical shape would be suitable for use with the disc repair device 90 of FIG. 7.

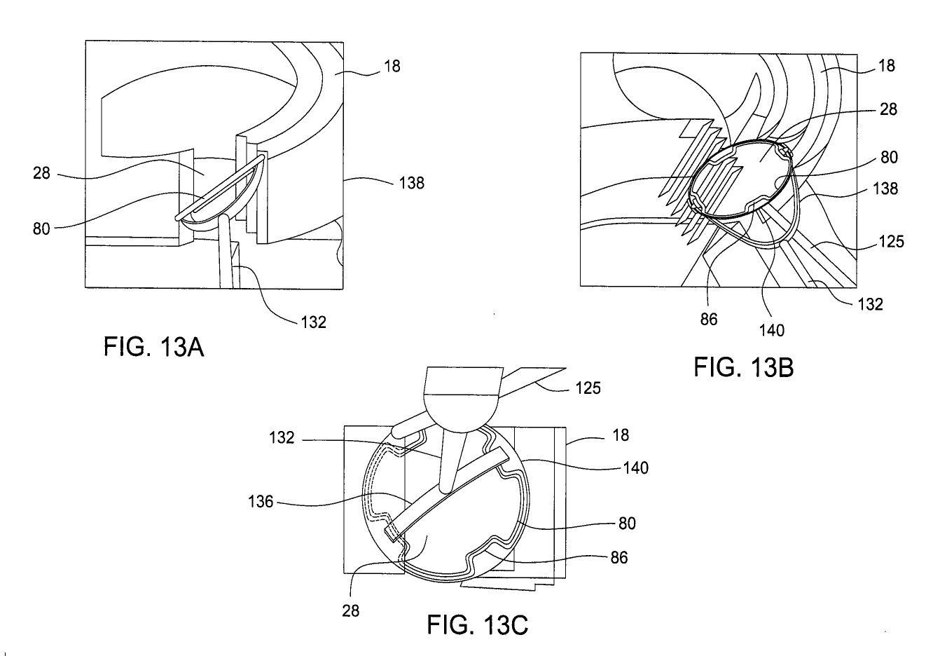

Various steps or acts of another method of implanting a disc repair device by use of the delivery instrument 130 are illustrated in FIGS. 13A-13C. For ease of insertion into the disc void 28, the holder 140 and the engaged disc repair device 80 are rotated such that the planar dimension of the repair device 80 is positioned to enter defect 28 inline with its greatest aspect or crosswise dimension (i.e., with a low profile), as illustrated in FIG. 13A. When reaching the target implant site, pre-cut or otherwise, the holder 140 is rotated such that the implant device 80 is positioned parallel to/inline with the implant site, as illustrated in FIG. 13C. This may be facilitated using a separate tool such as tool 125 (as illustrated in FIG. 13B) or one that is integrated with the delivery instrument 130. Once properly aligned within the implant site, the ring 140 may be slightly deflected or twisted out of plane to release the frictional hold on the implant device 80. The freed implant device 80 then readily inserts within the pre-cut space and becomes lodged within the disc endplates.

The various manipulations of the implant device 80 during delivery may be accomplished by use of tool 125 or the like; however, there are a number of other ways in which manipulation, rotation and/or release of the implant device from the delivery tool may be accomplished which can be readily appreciated by those skilled in the art. For example, the holder 140 may be slightly diametrically expanded to release its hold on the implant device. This action may be integrated into the instrument 130 whereby an actuator is activated by a user to cause expansion of the holder and release of the implant device.

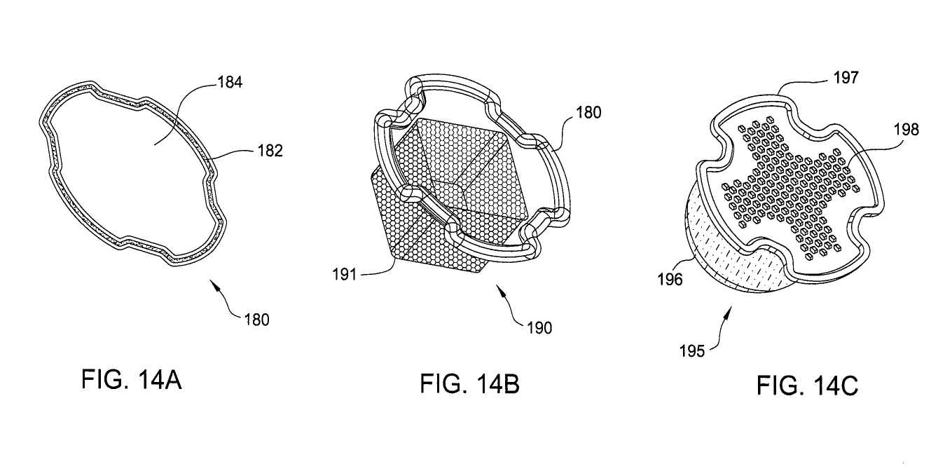

FIGS. 14A-C illustrate additional embodiments of implantable disc repair devices of the present invention. FIG. 14A shows a polymer coated ring-type implant device 180 similar to the implant device 80 of FIG. 6A. The device 180 includes a frame 182 and a polymer material 84 which is held in a relatively taut state by the frame 182 and expands across the frame 182 to define a planar implant device. The frame 182 may be made of a flexible wire such as NITINOL wire, a semi-rigid or rigid metal, or polymer wire having a diameter from about 0.05 mm to about 2 mm and more typically from about 0.1 mm to about 1 mm. FIG. 14B illustrates an implant device 190 having the ring-type implant device 180 of FIG. 14A configured with a plug 191. The plug 191 may have a porous matrix and may be made of a material that provides the in-growth and/or through-put characteristics as described above. Exemplary plug materials include foam, collagen fiber, biodegradable material, polyurethane, polyethylene, non-reactive/inert material, and combinations thereof. The matrix in the plug 191 may act as a scaffolding to promote in-growth of tissues. The plug may additionally include a drug coating, growth factors, or other drugs or chemicals to promote the healing process. FIG. 14C illustrates a polymer-coated plate-type implant device 195 configured with a plug 196. The implant device 195 may include a plate 197 having one or more apertures 198 and/or a polymer coating. The implant devices 180, 190, 195 may have any suitable shape (e.g., circular, oval, elliptical, rectangular, etc.) provided that the axial and transverse dimensions (as defined above) are sufficient to repair the defective annulus. The plug 196 may initially be retained in a compressed condition until implant device 195 has been implanted. In one embodiment, sutures may be used to restrain the plug 196. After implantation, the sutures may be broken to allow expansion of the plug 196 at the implant site. In one embodiment, at least one of the axial and transverse dimensions of the implant device is larger than the axial and transverse dimensions of the annulus defect.

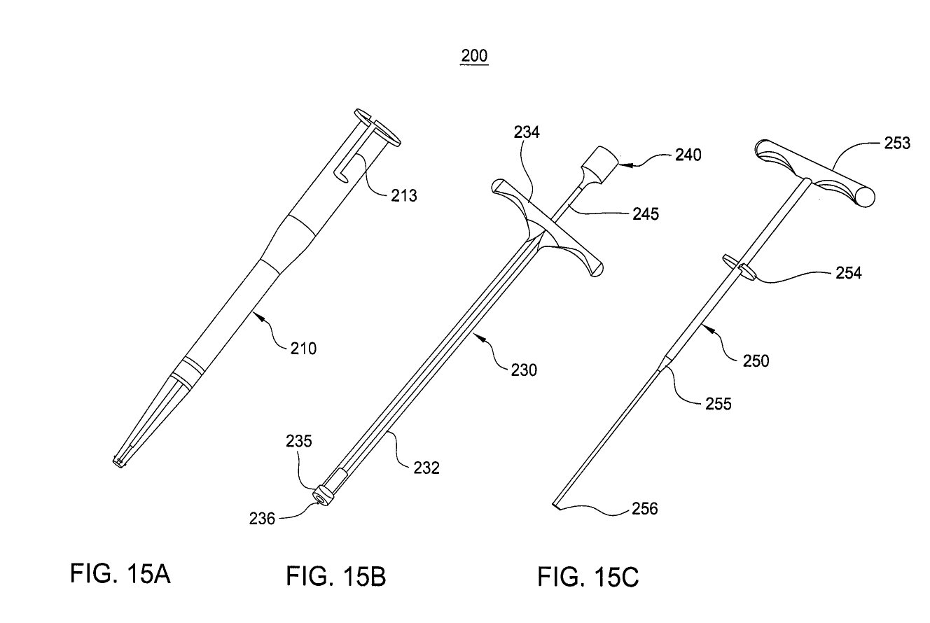

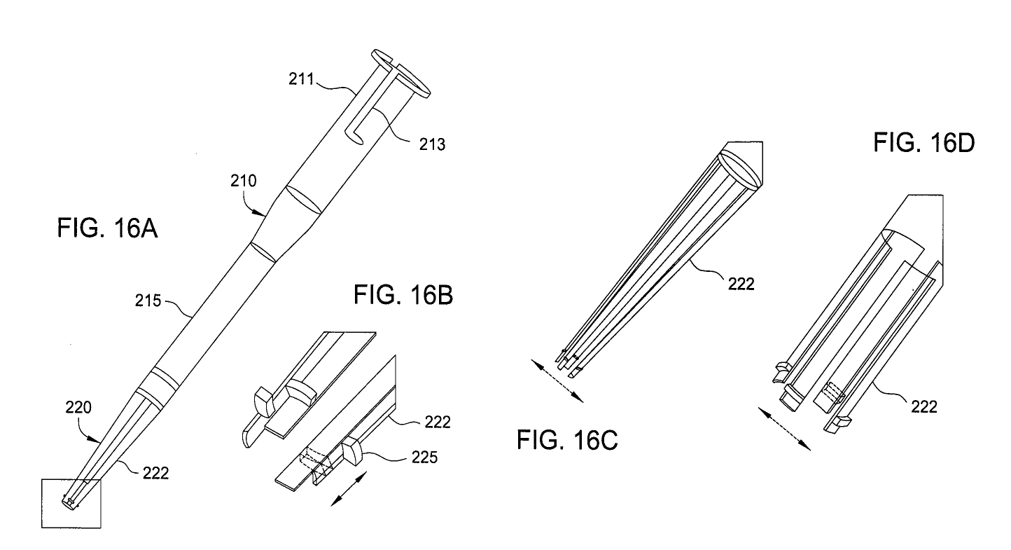

FIGS. 15A-15C illustrate various components of an implant delivery system 200 of the present invention, where FIG. 15A shows a dilator 210, FIG. 15B shows an implant holder 230, and FIG. 15C shows a cutting device 250. Referring now to FIGS. 16A-16D, the dilator 210 includes a tubular body 215 having a tool receiving end 211 and a tool delivery end 220. The tool receiving end 211 may be sized to accommodate a tool such as the implant holder 230 and the cutter 250. The dilator 210 may also include a guide rail 213 to guide the movement of the tool. The tool delivery end 220 may include four prongs 222 extending from the tubular body 215. One or more defect guides 225 may be positioned on the exterior of the prongs 222 to facilitate positioning of the dilator 210 relative to the annulus, as shown in FIG. 16B. For example, the defect guide 225 may be positioned about 2 mm away from the distal end of the prongs 222 such that the implant device may be delivered 2 mm deep into the annulus. The prongs 222 are configured such that the distal end of the prongs 222 has a smaller diameter than the proximal end. To accommodate the implant device, the prongs 222 are adapted to expand as the implant device is urged through the prongs 222 toward the annulus. In this respect, the prongs 222 may be used dilate the defect 28 to accommodate the implant device. FIG. 16C shows the prongs 222 before expansion and FIG. 16D shows the prongs 222 after expansion. In one embodiment, the unexpanded diameter of the prongs 222 is about 4 mm and the expanded diameter of the prongs 222 is about 8 mm. However, it must be noted that the prongs 222 may be adapted to accommodate an implant device of any size. Further, the dilator 210 many include any number of prongs to achieve the delivery of the implant device.

FIG. 15B shows the implant holder 230 coupled to an implant pusher 240. The implant holder 230 includes an elongated tubular shaft 232 having a handle portion 234 at a proximal end and an implant holding mechanism 235 at a distal end. The implant holding mechanism 235 may include a holding surface for receiving the implant device and implant guides 236 protruding from the holding surface adapted to engage the recesses 86 in the implant device. The implant guides 236 provide proper orientation of the implant device and may be sized to hold the implant device in place during delivery. The elongated tubular shaft 232 is sized for insertion into the tubular body 215 of the dilator 210. The implant pusher 240 includes an elongated shaft 245 sized for insertion into the tubular shaft 232 of the implant holder 230. The elongated shaft 245 may be inserted through the implant holder 230 in order to release the implant device from the implant holder 230.

FIG. 15C shows an embodiment of a cutting device 250 suitable for use with the dilator 210. The cutting device 250 includes an elongated shaft 255 insertable through the dilator 210, a handle portion 253 at a proximal end, and a cutter 256 at a distal end. In one embodiment, the cutter 256 is in the form of a blade and is positioned at about a right angle relative to the elongated shaft 255. The cutting device 250 may also include a guide stop 254 position on the shaft 255 to prevent over insertion of the cutting device 250 into the dilator 210.

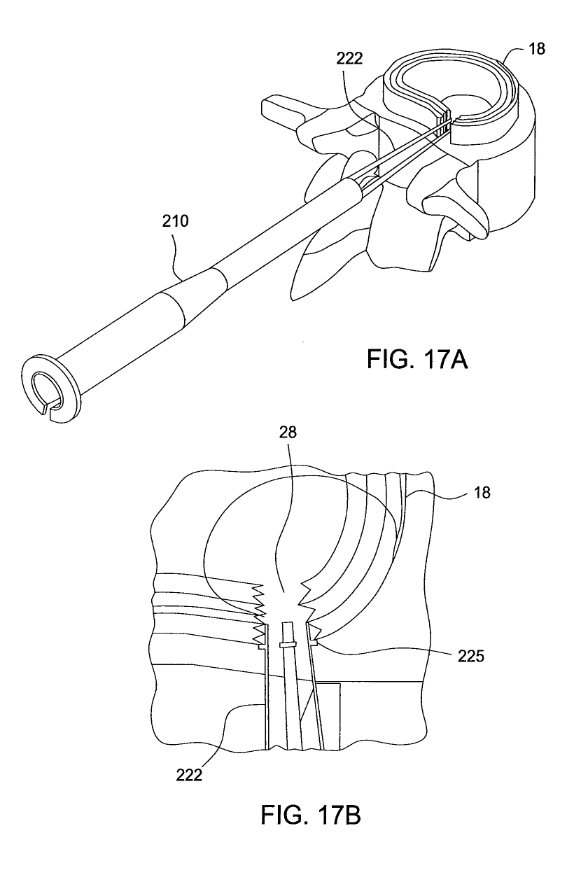

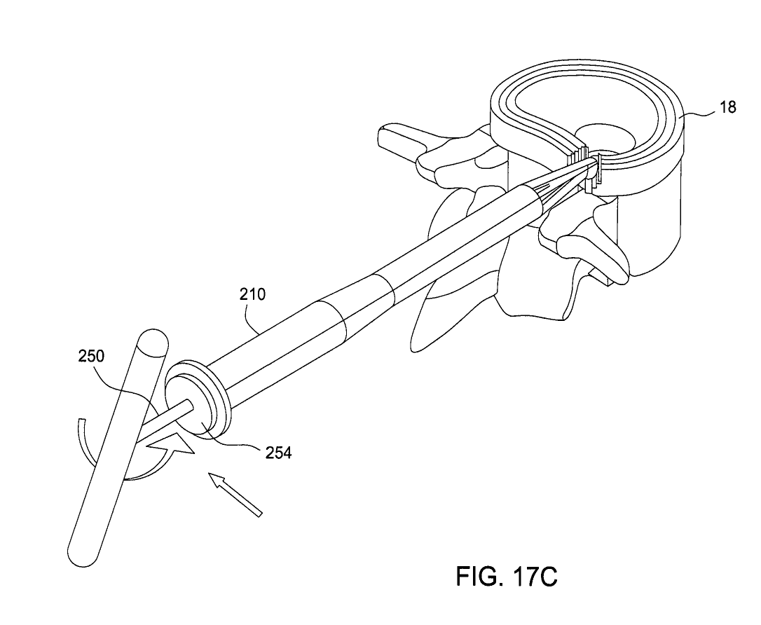

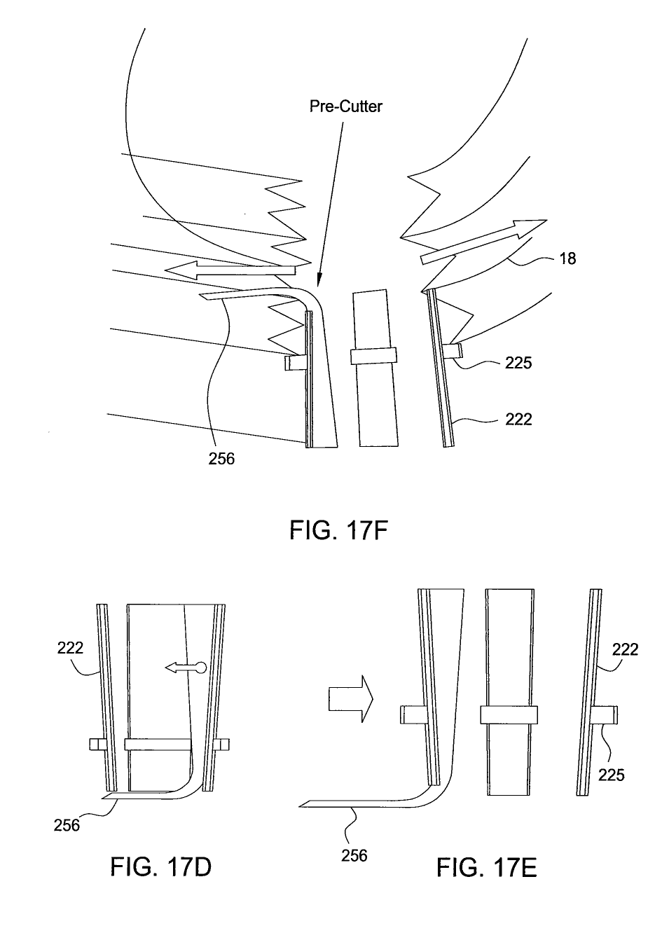

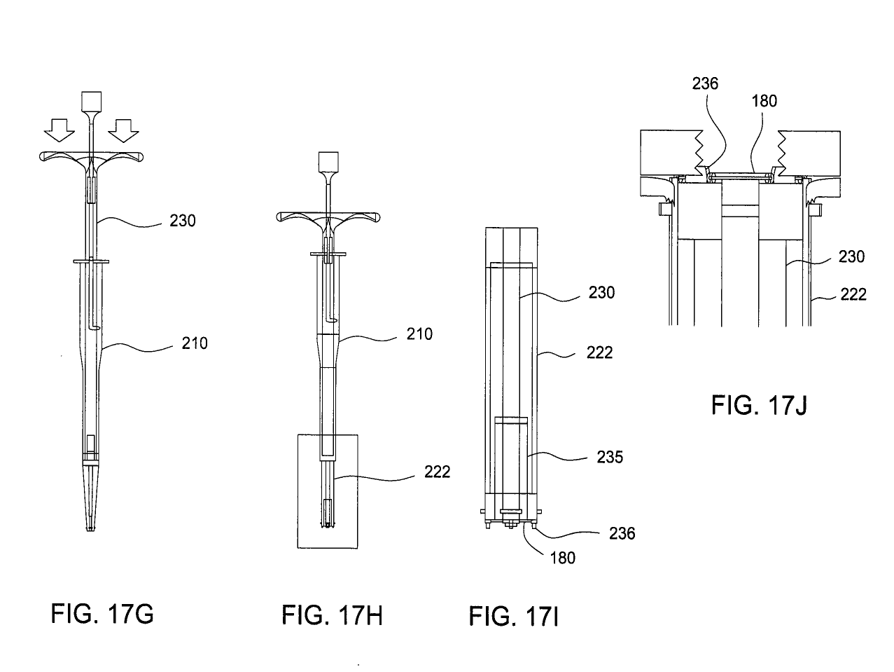

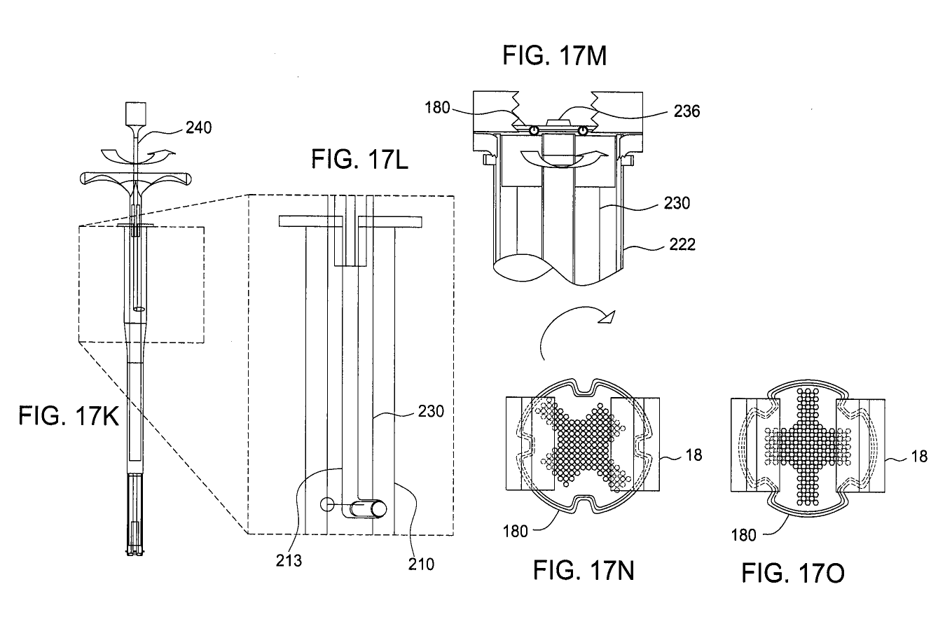

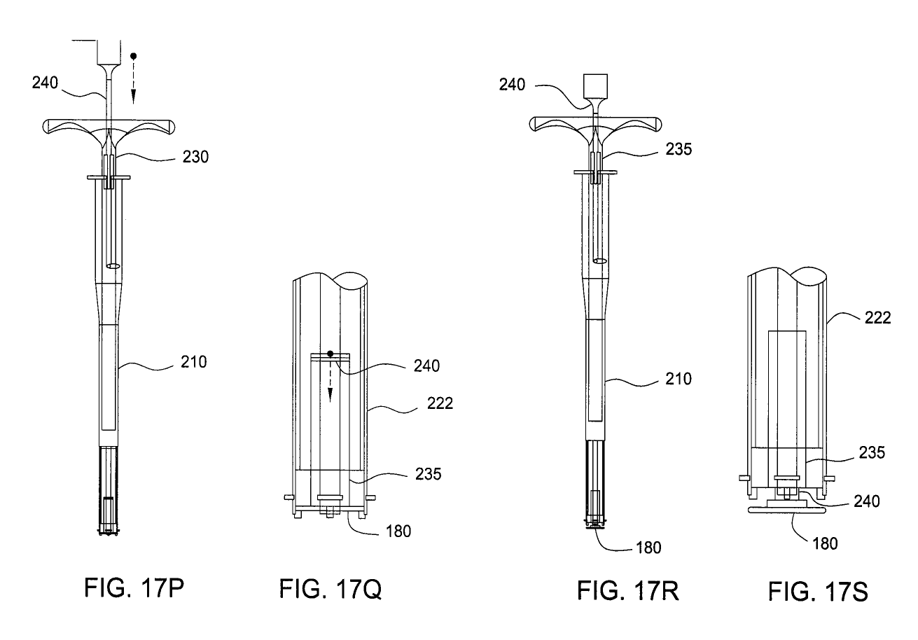

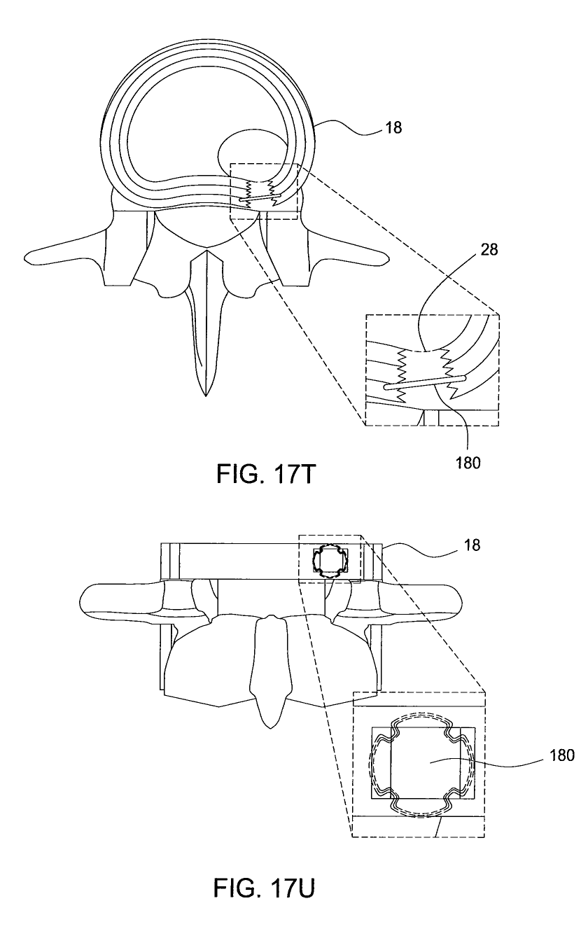

FIGS. 17A-17F illustrate various acts for implanting the disc repair device of FIG. 14A using the system of FIGS. 15A-15C. FIG. 17A shows the introduction of the dilator 210 into the annulus defect. FIG. 17B is an exploded partial view of FIG. 17A. In FIG. 17B, it can be seen that the defect guides 225 of the prongs 222 are urged against the annulus 18, which provides confirmation that the dilator 210 is properly positioned. It can also be seen that the prongs 222 are partially inserted into the annular defect 28. After the dilator 210 is introduced, the cutting device 250 may be inserted into the dilator 210, as illustrated in FIG. 17C. The cutting device 250 is inserted until the guide stop 254 is urged against the dilator 210. At this point, the cutter 256 may be pivoted to a cutting position such that the cutter 256 extends out of the prongs 222 and that the prongs 222 are expanded. FIGS. 17D-17E shows the cutter 256 transitioning to the cutting position and the prongs 222 in the expanded position. Then, the cutting device 250 is rotated to separate or cut between the lamellae of the annulus 18, as illustrated in FIG. 17F. To deliver the implant device 180, the implant device 180 is initially loaded onto the implant holding mechanism 235 of the implant holder 230. The loaded implant holder 230 is then inserted into the dilator 210, as shown in FIG. 17G. As the implant holder 230 is inserted, the prongs begin to expand to the larger size of the implant holding mechanism 235. The expansion dilates the defect opening 28 and allows the implant device 180 to be moved toward the defect opening 28. FIGS. 17H-17I show the implant device 180 positioned at the distal end of the prongs 222. FIG. 17J shows the implant device 180 positioned between two layers of lamellae. Thereafter, the implant holder 230 is rotated about a quarter turn such that the non-recessed outer portions of the implant device 180 are positioned axially and transversely relative to the annulus. To facilitate rotation, the implant holder 230 may include a key that is inserted in the guide rail 213, as shown in FIGS. 17K-17L. The key and guide rail 213 act to limit the rotation of the implant holder 230. FIG. 17J and FIG. 17M are top views of the implant device 180 before and after rotation, respectively. FIG. 17N and FIG. 17O are side views of FIG. 17J and FIG. 17M, respectively. It can be seen in FIG. 17O that portions of the implant device 180 are lodged in the intervertebral discs. Once properly aligned within the implant site, the implant device 180 may be released from the implant holder 230. The implant pusher 240 is urged toward the implant device 180 (see FIGS. 17P-Q) and pushes implant device away from the implant holder 230 (see FIGS. 17R-S), thereby causing the release of the implant device 180. FIGS. 17T-17U are top view and side view of the implant device 180 fully implanted at the implant site. It can be seen that the implant device 180 is positioned between two layers of lamellae and straddles the annular defect 28. Although embodiments of the implant device are shown positioned within the annular layers, it must be noted that one or more of the implant devices described herein may also be positioned in the sub-annular layer. Additionally, the implant procedure may be performed without rotation the implant device 180 after insertion. In this respect, the implant device may be inserted with the proper orientation before release or released from its inserted orientation.

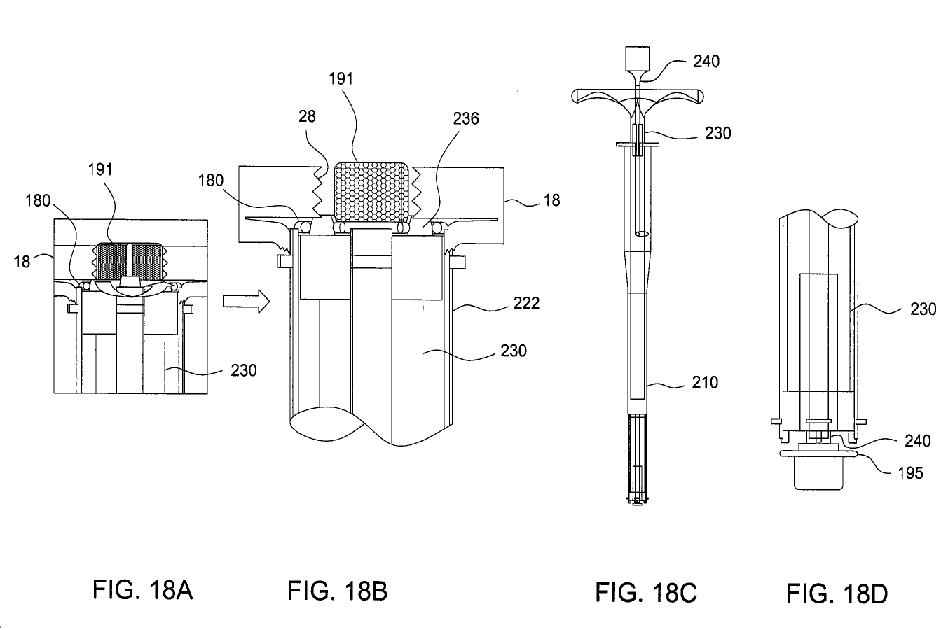

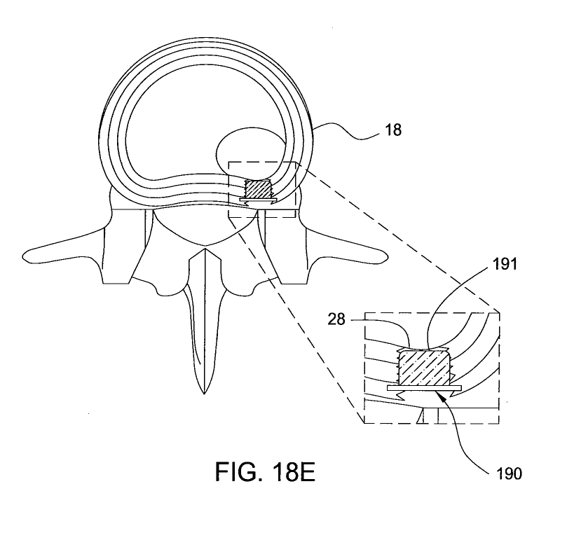

FIGS. 18A-E illustrate various steps or acts of implanting the disc repair device 190 of FIG. 14B using the delivery system shown in FIGS. 15A-C. In one embodiment, the process of FIG. 17 may be followed to implant the disc repair device 190 and will not be discussed in detail for clarity purposes. After the dilator 210 is introduced into the annulus, the cutting device 250 is used to cut a space in the lamellae for the repair device 190. The repair device 190 is then loaded onto the implant holder 230 and delivered to the implant site. As shown in FIG. 18A, the ring 180 of the repair device 190 is positioned in the annular layers. The plug 191 of the repair device 190 fills a large portion of the defect 28. Thereafter, the implant holder 230 is rotated about a quarter turn such that the non-recessed portions of the implant device 190 are positioned axially and transversely relative to the annulus. FIG. 18B shows the orientation of the implant device 190 after rotation. Once properly aligned within the implant site, the implant device 190 may be released from the implant holder 230. The implant pusher 240 is urged toward the implant device 190 and pushes the implant device 190 away from the implant holder 230 (see FIGS. 18C-D), thereby causing the release of the implant device 190. FIG. 18E is a top view of the implant device 190 fully implanted at the implant site. It can be seen that the implant device 190 is positioned between two layers of lamellae and straddles the annular defect 28. The foam matrix of the plug 191 may facilitate the in-growth of tissue by providing a scaffold platform for growth. After a period of time, tissue growth attached to the plug 191 may act as an additional securing feature to retain the implant device 190 in position.

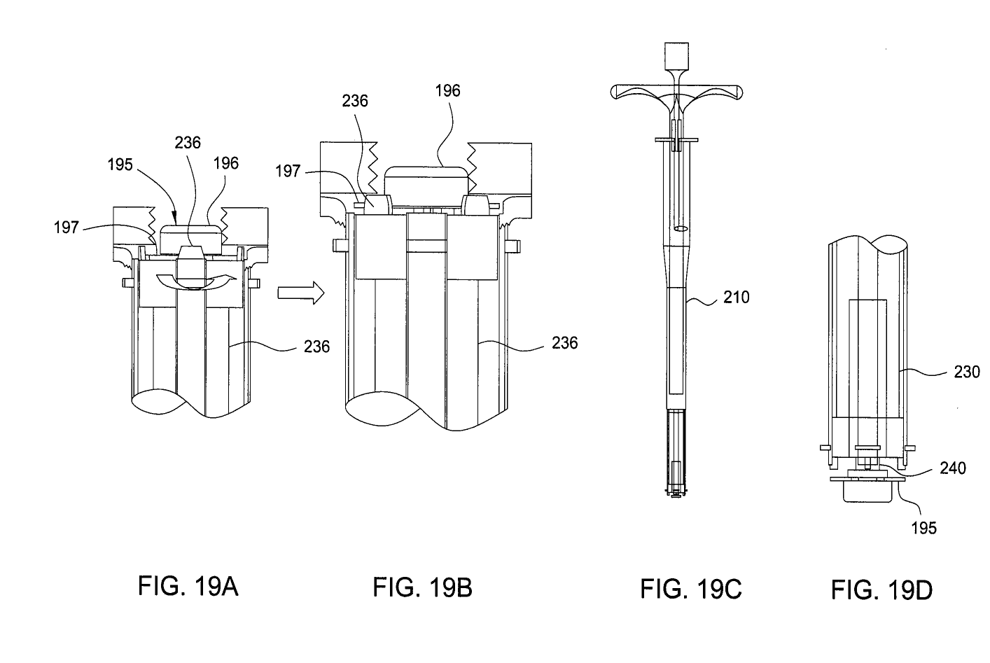

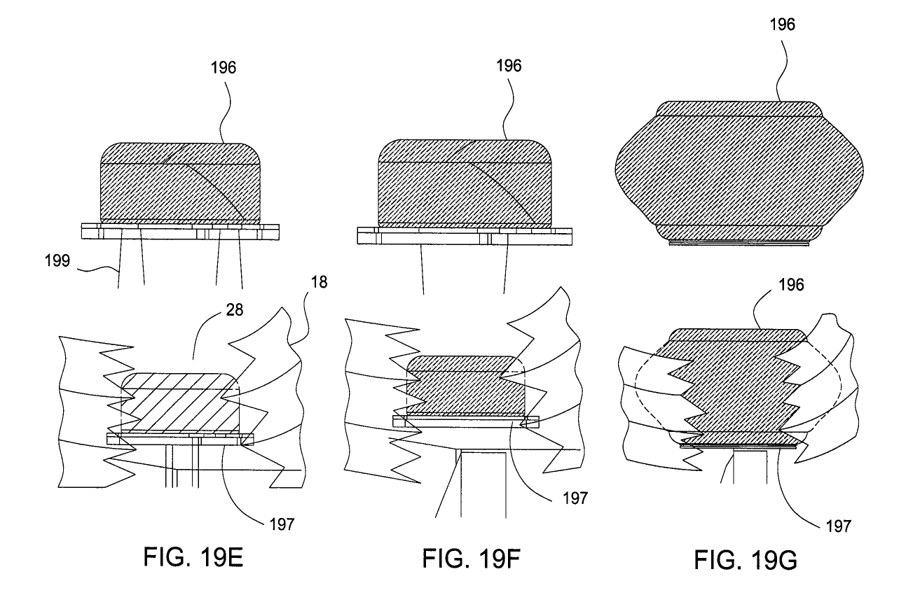

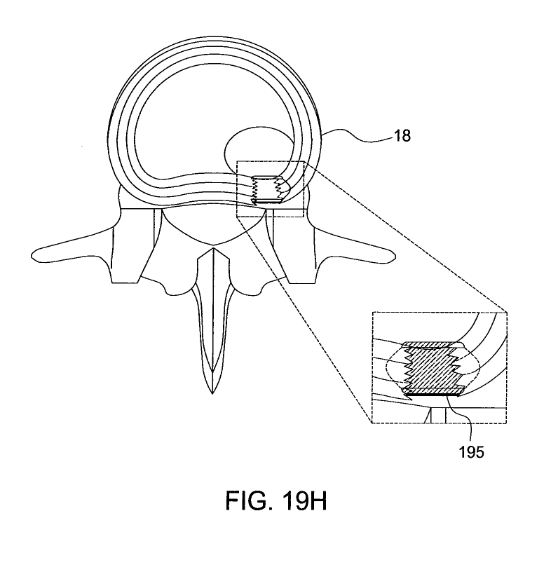

FIGS. 19A-H illustrate various steps or acts of implanting the plate-type disc repair device 195 of FIG. 14C using the delivery system shown in FIGS. 15A-C. In one embodiment, the process of FIG. 17 may be followed to implant the disc repair device 195 and will not be discussed in detail for clarity purposes. After the dilator 210 is introduced into the annulus, the cutting device 250 is used to cut a space in the lamellae for the repair device 195. The repair device 195 is then loaded onto the implant holder 230 and delivered to the implant site. As shown in FIG. 19A, the plate 197 of the repair device 195 is positioned in the annular layers and the plug 196 is positioned in the defect 28. Thereafter, the implant holder 230 is rotated about a quarter turn such that the non-recessed portions of the implant device 195 are positioned axially and transversely relative to the annulus. FIG. 19B shows the orientation of the implant device 195 after rotation. Once properly aligned within the implant site, the implant device 195 may be released from the implant holder 230. The implant pusher 240 is urged toward the implant device 195 and pushes the implant device 195 away from the implant holder 230 (see FIGS. 19C-D), thereby causing the release of the implant device 195. FIG. 19E is a top view of the implant device 190 fully implanted at the target site. It can be seen that the implant device 190 is positioned between two layers of lamellae and straddles the annular defect 28. Further, the pre-attached sutures 199 are in a taut condition and holding the plug 196 is an unexpanded state. Then, the sutures 199 are cut to allow expansion of the plug 196, as shown in FIG. 19F. Upon cutting the sutures 199, subsequent expansion of the implant device causes the plug 196 to engage the defect 28, thereby providing an additional retention measure for the implant device 195, as illustrated in FIG. 19G. FIG. 19H is a top view of the implant device 195 fully expanded in the defect 28. In one embodiment, the plug 196 made be manufactured from a polyurethane foam. The foam 196 may facilitate the in-growth of tissue by providing a scaffold platform for growth. It is contemplated that a portion of the various plugs described herein may extend, due to either the natural shape or through expansion, into the nucleus portion of the disc. In addition, the plugs may expand sufficiently to substantially or partially conform to the defect area 28 or any suitable shape. After a period of time, tissue growth attached to the foam 196 may act as another securing feature to retain the implant device 190 in position. In another embodiment, the plug 196 may be provided with a drug coating or growth factors promote the healing process.

It should be noted that any of the above-described acts, steps or procedures, including but not limited to cannulation of the target area, removal of the affected portion of the disc, forming a pre-cut target implant space within the disc, implantation of the subject implants within the target implant site, and/or adjustment or readjustment of the implant may be facilitated by way of a scope integrated within a cutting and/or delivery instrument or by way of various visualization techniques including but not limited to real time fluoroscopy, CT scanning or MR imaging, or a combination of preoperative CT or MR images superimposed onto a real time image tracking device, which are well known in the surgical arts.

Further, it is understood that the subject methods may all comprise the act of providing a suitable device. Such provision may be performed by the end user. In other words, the “providing” (e.g., a disc augmentation device) merely requires the end user obtain, access, approach, position, set-up, activate, power-up or otherwise act to provide the requisite device in the subject method. Methods recited herein may be carried out in any order of the recited events which is logically possible, as well as in the recited order of events.

The subject devices and instrumentation may be provided in the form of a kit which includes at least one disc repair device of the present invention. A plurality of such devices may be provided where the devices have the same or varying sizes and shapes and are made of the same or varying materials. The kits may further include instruments and tools for pre-cutting the implant site and implanting the subject devices, including but not limited to those described above as well as cannulas, trocars, scopes, sheaths, etc. Instructions for implanting the subject devices and for using the above-described instrumentation may also be provided with the kits.

In one embodiment, an implantable device for repairing a defective area of an annulus of an intervertebral disc includes a planar structure having a dimension greater than the defective area wherein at least a portion of the implantable device extends beyond the defective area upon implantation within the defective area.

In another embodiment, a system of treating an intervertebral disc annulus includes an implantable device; a dilator for dilating an opening in the annulus; and a holder for holding the implantable device. In yet another embodiment, the system further includes a cutting device for cutting the annulus.

In another embodiment, a system of treating an intervertebral disc annulus includes an implantable device and a delivery instrument for holding the implantable device and delivering the implantable device to an opening in the annulus, wherein the delivery instrument is adapted to deliver the implantable device in its natural shape. In yet another embodiment, thee delivery instrument includes a shaft and a device holding mechanism. In yet another embodiment, the device holding mechanism includes at least two arms adapted to engage an outer perimeter of the implantable device.

In another embodiment, a method of treating a defective area of an intervertebral disc annulus situated between upper and lower vertebra comprises providing a device comprising a planar structure having a dimension greater than the defective area; positioning the device in the defective area; and lodging the device within the defective area, wherein at least a portion of the device extends beyond the defective area.

In one or more of the embodiments described herein, the implantable device includes a plug.

In one or more of the embodiments described herein, the plug is made of an expandable material.

In one or more of the embodiments described herein, at least a portion of the plug may extend into the nucleus.

In one or more of the embodiments described herein, the plug is expandable to conform to at least a portion of the defect.

In one or more of the embodiments described herein, the implant procedure includes dilating the defective area.

In one or more of the embodiments described herein, the device has at least one dimension that is greater than a natural disc height.

In one or more of the embodiments described herein, the implant device is delivered in its natural configuration.

In one or more of the embodiments described herein, the implantable device is configured to prevent material within the disc from escaping.

In one or more of the embodiments described herein, the implantable device is configured for implantation between two adjacent lamellae of the annulus.

In one or more of the embodiments described herein, the implant device includes a blade portion.

In one or more of the embodiments described herein, the implant device includes a cutting structure.

In one or more of the embodiments described herein, the implant device includes an anchor.

In one or more of the embodiments described herein, the implant device includes the anchor is biased away from a surface of the implantable device.

In one or more of the embodiments described herein, the implant device includes a foam material.

In one or more of the embodiments described herein, the foam material is selected from the group consisting of collagen fiber, biodegradable material, polyurethane, polyethylene, non-reactive/inert material, and combinations thereof.

In one or more of the embodiments described herein, the implant device includes comprising a drug additive.

In one or more of the embodiments described herein, the implant device is configured to receive sutures which hold the plug in a compressed condition.

It must be noted that as used herein and in the appended claims, the singular forms “a”, “an”, and “the” include plural referents unless the context clearly dictates otherwise. Thus, for example, reference to “a device” may include a plurality of such devices and reference to “the material” includes reference to one or more materials and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An implantable device for repairing a defective area of an annulus of an intervertebral disc, the device comprising:
a planar structure having a dimension greater than the defective area wherein at least a portion of the implantable device extends beyond the defective area upon implantation within the defective area.

2. The implantable device of claim 1, wherein the implantable device is configured to prevent material within the disc from escaping.

3. The implantable device of claim 1, wherein the device has at least one dimension that is greater than a natural disc height.

4. The implantable device of claim 1, wherein the implantable device has an elongated configuration.

5. The implantable device of claim 1, wherein the implantable device has a circular configuration.

6. The implantable device of claim 1, wherein the implantable device is configured for implantation between two adjacent lamellae of the annulus.

7. The implantable device of claim 1, further comprising a blade portion.

8. The implantable device of claim 1, further comprising a cutting structure.

9. The implantable device of claim 1, further comprising an anchor.

10. The implantable device of claim 8, wherein the anchor is biased away from a surface of the implantable device.

11. The implantable device of claim 10, further comprising a cutting structure adapted to maintain the anchor in an unbiased condition.

12. The implantable device of claim 1, further comprising a plug extending from the planar structure.

13. The implantable device of claim 12, wherein the plug comprises a foam material.

14. The implantable device of claim 13, wherein the foam material is selected from the group consisting of collagen fiber, biodegradable material, polyurethane, polyethylene, non-reactive/inert material, and combinations thereof.

15. The implantable device of claim 12, further comprising a drug additive.

16. The implantable device of claim 15, wherein the drug additive comprises a drug coating.

17. The implantable device of claim 12, wherein the planar structure is configured to receive sutures which hold the plug in a compressed condition.

18. The implantable device of claim 12, wherein the plug is expandable.

19. A system of treating an intervertebral disc annulus, the system comprising:
an implantable device;
a dilator for dilating an opening in the annulus; and
a holder for holding the implantable device.

20. The system of claim 19, further comprising a cutting device for cutting the annulus.

21. The system of claim 20, wherein the cutting device is insertable into the dilator.

22. The system of claim 21, wherein the cutting device is rotatable in the dilator.

23. The system of claim 19, wherein the dilator comprises one or more prongs.

24. The system of claim 23, wherein the expandable prongs are expandable.

25. The system of claim 19, wherein the holder is insertable into the dilator to deliver the implantable device to the opening.

26. The system of claim 25, further comprising an implant pusher for releasing the implantable device from the holder.

27. A system of treating an intervertebral disc annulus, the system comprising:
an implantable device; and
a delivery instrument for holding the implantable device and delivering the implantable device to an opening in the annulus, wherein the delivery instrument is adapted to deliver the implantable device in its natural shape.

28. The system of claim 27, wherein the delivery instrument comprises a shaft and a device holding mechanism.

29. The system of claim 28, wherein the device holding mechanism comprises at least two arms adapted to engage an outer perimeter of the implantable device.

30. The system of claim 29, wherein the device holding mechanism further comprises a ring pivotally coupled to the at least two arms.

31. A method of treating a defective area of an intervertebral disc annulus situated between upper and lower vertebra, the method comprising:
providing a device comprising a planar structure having a dimension greater than the defective area;
positioning the device in the defective area; and
lodging the device within the defective area, wherein at least a portion of the device extends beyond the defective area.

32. The method of claim 31, further comprising dilating the defective area.

33. The method of claim 32, further comprising cutting at least a portion of annulus prior to lodging the device.

34. The method of claim 31, further comprising providing the device with a foam material.

35. The method of claim 34, further comprising allowing the foam material to expand.

36. The method of claim 35, wherein the foam material substantially conforms to a shape of the defective area.

37. The method of claim 35, wherein at least a portion of the foam material expands into the nucleus.

38. The method of claim 31, further comprising cutting at least a portion of annulus prior to lodging the device.
